(19) United States
(12) Patent Application Publication
(10) Pub. No.: US 2018/0050040 A1
(43) Pub. Date: Feb. 22, 2018

(54) USE OF 4-(4-FLUORO-2-METHOXYPHENYL)-N--1,3,5-TRIAZIN-2-AMINE FOR TREATING LYMPHOMAS

(71) Applicants: Bayer Pharma Aktiengesellschaft, Berlin (DE); Public University Corporation Nagoya City University, Nagoya (JP)

(72) Inventors: Arne SCHOLZ, Berlin (DE); Takashi ISHIDA, Nagoya-Shi (JP)

(21) Appl. No.: 15/560,969

(22) PCT Filed: Mar. 21, 2016
(86) PCT No.: PCT/EP2016/056112

§ 371 Date: Sep. 22, 2017

(30) Foreign Application Priority Data
Mar. 24, 2015 (EP) 15160590.4

(51) Int. Cl. A61K 31/53

(52) U.S. Cl. CPC A61K 31/53

(57) ABSTRACT

The present invention relates to 4-(4-Fluoro-2-methoxyphenyl)-N-{3-[(S-methylsulfonimidoyl)methyl]phenyl}-1,3,5-triazin-2-amine (compound A), more particularly (+)-4-(4-Fluoro-2-methoxyphenyl)-N-{3-[(S-methylsulfonimidoyl)methyl]phenyl}-1,3,5-triazin-2-amine (compound A′), for use in treating lymphoma, preferably diffuse large B-cell lymphoma (DLBCL) mantle cell lymphoma, follicular lymphoma, diffuse large B-cell lymBurkitt's lymphoma, adult T-cell lymphoma (ATL) and Hodgkin's lymphoma, more preferably DLBCL and ATL.

The present invention relates to the use of 4-(4-Fluoro-2-methoxyphenyl)-N-{3-[(S-methylsulfonimidoyl)methyl]phenyl}-1,3,5-triazin-2-amine (compound A), more particularly (+)-4-(4-Fluoro-2-methoxyphenyl)-N-{3-[(S-methylsulfonimidoyl)methyl]phenyl}-1,3,5-triazin-2-amine (compound A′, for treating lymphoma, preferably diffuse large B-cell lymphoma (DLBCL), mantle cell lymphoma, follicular lymphoma, diffuse large B-cell lymphoma, Burkitt's lymphoma, adult T-cell lymphoma (ATL) and Hodgkin's lymphoma, more preferably DLBCL and ATL.

The family of cyclin-dependent kinase (CDK) proteins consists of members that are key regulators of the cell division cycle (cell cycle CDK's), that are involved in regulation of gene transcription (transcriptional CDK's), and of members with other functions. CDKs require for activation the association with a regulatory cyclin subunit. The cell cycle CDKs CDK1/cyclin B, CDK2/cyclin A, CDK2/cyclinE, CDK4/cyclinD, and CDK6/cyclinD get activated in a sequential order to drive a cell into and through the cell division cycle. The transcriptional CDKs CDK9/cyclin T and CDK7/cyclin H regulate the activity of RNA polymerase II via phosphorylation of the carboxy-terminal domain (CTD). Positive transcription factor b (P-TEFb) is a heterodimer of CDK9 and one of four cyclin partners, cyclin T1, cyclin K, cyclin T2a or T2b.

Whereas CDK9 (NCBI GenBank Gene ID 1025) is exclusively involved in transcriptional regulation, CDK7 in addition participates in cell cycle regulation as CDK-activating kinase (CAK).

Transcription of genes by RNA polymerase II is initiated by assembly of the pre-initiation complex at the promoter region and phosphorylation of Ser 5 and Ser 7 of the CTD by CDK7/cyclin H. For a major fraction of genes RNA polymerase II stops mRNA transcription after it moved 20-40 nucleotides along the DNA template. This promoter-proximal pausing of RNA polymerase II is mediated by negative elongation factors and is recognized as a major control mechanism to regulate expression of rapidly induced genes in response to a variety of stimuli (Cho et al., Cell Cycle 2010, 9, 1697). P-TEFb is crucially involved in overcoming promoter-proximal pausing of RNA polymerase II and transition into a productive elongation state by phosphorylation of Ser 2 of the CTD as well as by phosphorylation and inactivation of negative elongation factors.

Activity of P-TEFb itself is regulated by several mechanisms. About half of cellular P-TEFb exists in an inactive complex with 7SK small nuclear RNA (7SK snRNA), La-related protein 7 (LARP7/PIP7S) and hexamethylene bis-acetamide inducible proteins 1/2 (HEXIM1/2, He et al., Mol. Cell 2008, 29, 588). The remaining half of P-TEFb exists in an active complex containing the bromodomain protein Brd4 (Yang et al., Mol. Cell 2005, 19, 535). Brd4 recruits P-TEFb through interaction with acetylated histones to chromatin areas primed for gene transcription. Through alternately interacting with its positive and negative regulators, P-TEFb is maintained in a functional equilibrium: P-TEFb bound to the 7SK snRNA complex represents a reservoir from which active P-TEFb can be released on demand of cellular transcription and cell proliferation (Zhou & Yik, Microbiol. Mol. Biol. Rev. 2006, 70, 646). Furthermore, the activity of P-TEFb is regulated by posttranslational modifications including phosphorylation/de-phosphorylation, ubiquitination, and acetylation (reviewed in Cho et al., Cell Cycle 2010, 9, 1697).

Deregulated CDK9 kinase activity of the P-TEFb heterodimer is associated with a variety of human pathological settings such as hyper-proliferative diseases (e.g. cancer), virally induced infectious diseases or cardiovascular diseases.

Cancer is regarded as a hyper-proliferative disorder mediated by a disbalance of proliferation and cell death (apoptosis). High levels of anti-apoptotic Bcl-2-family proteins are found in various human tumours and account for prolonged survival of tumour cells and therapy resistance. Inhibition of P-TEFb kinase activity was shown to reduce transcriptional activity of RNA polymerase II leading to a decline of short-lived anti-apoptotic proteins, especially Mcl-1 and XIAP, reinstalling the ability of tumour cells to undergo apoptosis. A number of other proteins associated with the transformed tumour phenotype (such as Myc, NF-kB responsive gene transcripts, mitotic kinases) are either short-lived proteins or are encoded by short-lived transcripts which are sensitive to reduced RNA polymerase II activity mediated by P-TEFb inhibition (reviewed in Wang & Fischer, Trends Pharmacol. Sci. 2008, 29, 302).

Many viruses rely on the transcriptional machinery of the host cell for the transcription of their own genome. In case of HIV-1 RNA polymerase II gets recruited to the promoter region within the viral LTR's. The viral transcription activator (Tat) protein binds to nascent viral transcripts and overcomes promoter-proximal RNA polymerase II pausing by recruitment of P-TEFb which in turn promotes transcriptional elongation. Furthermore, the Tat protein increases the fraction of active P-TEFb by replacement of the P-TEFb inhibitory proteins HEXIM1/2 within the 7SK snRNA complex. Recent data have shown that inhibition of the kinase activity of P-TEFb is sufficient to block HIV-1 replication at kinase inhibitor concentrations that are not cytotoxic to the host cells (reviewed in Wang & Fischer, Trends Pharmacol. Sci. 2008, 29, 302). Similarly, recruitment of P-TEFb by viral proteins has been reported for other viruses such as B-cell cancer-associated Epstein-Barr virus, where the nuclear antigen EBNA2 protein interacts with P-TEFb (Bark-Jones et al., Oncogene 2006, 25, 1775), and the human T-lymphotropic virus type I (HTLV-1), where the transcriptional activator Tax recruits P-TEFb (Zhou et al., J. Virol. 2006, 80, 4781).

Cardiac hypertrophy, the heart's adaptive response to mechanical overload and pressure (hemodynamic stress e.g. hypertension, myocardial infarction), can lead, on a long term, to heart failure and death. Cardiac hypertrophy was shown to be associated with increased transcriptional activity and RNA polymerase II CTD phosphorylation in cardiac muscle cells. P-TEFb was found to be activated by dissociation from the inactive 7SK snRNA/HEXIM1/2 complex. These findings suggest pharmacological inhibition of P-TEFb kinase activity as a therapeutic approach to treat cardiac hypertrophy (reviewed in Dey et al., Cell Cycle 2007, 6, 1856).

In summary, multiple lines of evidence suggest that selective inhibition of the CDK9 kinase activity of the P-TEFb heterodimer (=CDK9 and one of four cyclin partners, cyclin T1, cyclin K, cyclin T2a or T2b) represents an innovative approach for the treatment of diseases such as cancer, viral diseases, and/or diseases of the heart. CDK9 belongs to a family of at least 13 closely related kinases of which the subgroup of the cell cycle CDK's fulfils multiple roles in regulation of cell proliferation. Thus, co-inhibition of cell cycle CDK's (e.g. CDK1/cyclin B, CDK2/cyclin A, CDK2/cyclinE, CDK4/cyclinD, CDK6/cyclinD) and of CDK9 is expected to impact normal proliferating tissues such as intestinal mucosa, lymphatic and hematopoietic organs, and reproductive organs. To maximize the therapeutic margin of CDK9 kinase inhibitors, molecules with high selectivity towards CDK9 are therefore required.

CDK inhibitors in general as well as CDK9 inhibitors are described in a number of different publications: WO2008129070 and WO2008129071 both describe 2,4 disubstituted aminopyrimidines as CDK inhibitors in general. It is also asserted that some of these compounds may act as selective CDK9 inhibitors (WO2008129070) and as CDK5 inhibitors (WO2008129071), respectively, but no specific CDK9 IC50 (WO2008129070) or CDK5 IC50 (WO200812971) data is presented.

WO2008129080 discloses 4,6 disubstituted aminopyrimidines and demonstrates that these compounds show an inhibitory effect on the protein kinase activity of various protein kinases, such as CDK1, CDK2, CDK4, CDK5, CDK6 and CDK9, with a preference for CDK9 inhibition (example 80).

EP1218360 B1 describes triazin derivatives as kinase inhibitors, but does not disclose potent or selective CDK9 inhibitors.

WO2008079933 discloses aminopyridine and aminopyrimidine derivatives and their use as CDK1, CDK2, CDK3, CDK4, CDK5, CDK6, CDK7, CDK8 or CDK9 inhibitors.

WO2011012661 describes aminopyridine derivatives useful as CDK inhibitors.

Wang et al. (Chemistry & Biology 2010, 17, 1111-1121) describe 2-anilino-4-(thiazol-5-yl)pyrimidine transcriptional CDK inhibitors, which show anticancer activity in animal models.

WO2004009562 discloses substituted triazine kinase inhibitors. For selected compounds CDK1 and CDK 4 test data, but no CDK9 data is presented.

WO2004072063 describes heteroaryl (pyrimidine, triazine) substituted pyrroles as inhibitors of protein kinases such as ERK2, GSK3, PKA or CDK2.

WO2010009155 discloses triazine and pyrimidine derivatives as inhibitors of histone deacetylase and/or cyclin dependent kinases (CDKs). For selected compounds CDK2 test data is described.

WO2003037346 (corresponding to U.S. Pat. No. 7,618,968B2, U.S. Pat. No. 7,291,616B2, US2008064700A1, US2003153570A1) relates to aryl triazines and uses thereof, including to inhibit lysophosphatidic acid acyltransferase beta (LPAAT-beta) activity and/or proliferation of cells such as tumour cells.

WO2008025556 describes carbamoyl sulfoximides having a pyrimidine core, which are useful as kinase inhibitors. No CDK9 data is presented.

WO2002066481 describes pyrimidine derivatives as cyclin dependent kinase inhibitors CDK9 is not mentioned and no CDK9 data is presented.

WO2008109943 concerns phenyl aminopyri(mi)dine compounds and their use as kinase inhibitors, in particular as JAK2 kinase inhibitors. The specific examples focus on compounds having a pyrimidine core.

WO2009032861 describes substituted pyrimidinyl amines as JNK kinase inhibitors. The specific examples focus on compounds having a pyrimidine core.

WO2011046970 concerns amino-pyrimidine compounds as inhibitors of TBKL and/or IKK epsilon. The specific examples focus on compounds having a pyrimidine core.

WO2012160034 the compounds of the present invention. It is disclosed the compounds inhibit the cell proliferation of HeLa cells (cervical cancer), HeLa/MaTu/ADR cells (cervical cancer), NCI-H460 cells (non-small cell lung cancer), DU145 cells (hormone-independent human prostate cancer), Caco-2 cells (colorectal cancer) and B16F10 cells (melanoma).

The object of the present invention is to improve the treatment of lymphoma, preferably diffuse large B-cell lymphoma (DLBCL), mantle cell lymphoma, follicular lymphoma, diffuse large B-cell lymBurkitt's lymphoma, adult T-cell lymphoma (ATL) and Hodgkin's lymphoma, more preferably DLBCL and ATL.

### Treatment of Lymphomas

Malignant neoplasms of B-lymphocytes and T-lymphocytes can be broadly characterised as Hodgkin and non-Hodgkin lymphomas. Non-Hodgkin lymphomas, in turn, represent a large heterogeneous population of diseases each with distinct epidemiology, aetiology, and morphologic, immunophenotypic, and clinical features. The World Health Organisation (WHO) reclassified non-Hodgkin lymphomas in 2008 and this now better reflects our understanding of the disease entities and their relationship to the immune system (Jaffe E S. The 2008 WHO classification of lymphomas: implications for clinical practice and translational research. Hematology Am Soc Hematol Educ Program 2009:523-531).

DLBCL is an aggressive disease and the most common subtype of non-Hodgkin lymphoma accounting for up to 30% of newly diagnosed cases in the United States (Morton L M et al. Lymphoma incidence patterns by WHO subtype in the United States, 1992-2001. Blood. 2006; 107:265-76). The primary modality for advanced-stage DLBCL is combination chemoimmunotherapy, specifically R-CHOP (rituximab, cyclophosphamide, doxorubicin, vincristine and prednisone). The introduction of rituximab into this chemotherapeutic regime has been the cornerstone to consistent and meaningful improvements in the outcome of DLBCL patients. However, a subset of patients with advanced DLBCL do not respond favourably to, or relapse, following R-CHOP therapy. As a result, a variety of treatment approaches have been explored in an attempt to improve outcomes, including delivery of more chemotherapy cycles, dose-dense and alternative drug combinations and high-dose chemotherapy, followed by autologous stem cell transplant. However, there has been little evidence that these therapies have superior efficacy to R-CHOP.

Therapeutic targeting of the specific molecular pathways involved in the development of DLBCL may ultimately lead to improvement in patient outcomes. Several novel agents undergoing evaluation, both as single agents in the relapsed-disease setting and in combination with R-CHOP include immunomodulatory drugs (IMiDs), protein kinase C inhibitors, histone deacetylase inhibitors, proteasome inhibitors and mTOR (mammalian target of rapamycin) inhibitors (Boyle J et al. Improving Outcomes in Advanced DLBCL: Systemic Approaches and Radiotherapy. Oncology (Williston Park) 2014; 28(12): pii: 202929; Nastoupil L J et al. Diffuse Large B-Cell Lymphoma: Current Treatment Approaches. Oncology (Williston Park) 2012; 26(5):488-95).

Therefore, alternative therapies are needed for DLBCL (particularly for relapse or aggressive disease subsets) and for other lymphoma types.

Adult T-cell lymphoma (ATL), a peripheral T-cell neoplasm, is caused by human T-cell lymphotropic virus type-1 (HTLV-1). ATL is classified into four clinical subtypes, namely acute, lymphoma, chronic, and smoldering, according to criteria proposed by the Japan Lymphoma Study Group. ATL patients with acute and lymphoma subtypes have disease that shows an aggressive clinical course, whereas ATL patients with the chronic and smoldering subtypes survive longer. Thus, in general, dose intensified combination chemotherapies are recommended for ATL patients with acute and lymphoma subtypes, and a watch and wait policy is recommended for ATL patients with chronic and smoldering subtypes (J Clin Oncol. 2009; 27(3):453-459). The median survival times of ATL patients with acute (n=465) and lymphoma (n=156) subtypes, who were diagnosed between 1983 and 1987, were reported to be 6.2 and 10.2 months, respectively (Br J Haematol. 1991; 79(3):428-437). Furthermore, the median survival time of ATL patients with acute and lymphoma subtypes (n=807), who were diagnosed between 2000 and 2009 and did not receive an allogeneic hematopoietic stem cell transplantation, was reported to be 7.7 months (J Clin Oncol. 2012; 30(14):1635-1640). These results indicate that the disease has a very poor prognosis, and minimal progress has been achieved in the treatment of ATL in the 40 years since its discovery. Therefore, the development of novel treatment strategies for patients with ATL is an ongoing, urgent issue.

It has now been found that the compound 4-(4-Fluoro-2-methoxyphenyl)-N-{3-[(S-methylsulfonimidoyl)methyl]phenyl}-1,3,5-triazin-2-amine (compound A, formula (I)),

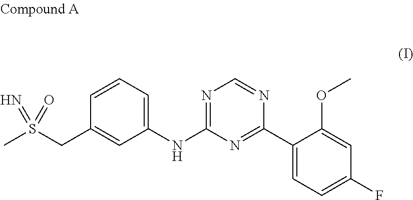 [SMILES: COc1cc(F)ccc1-c1ncnc(Nc2cccc(CS(C)(=N)=O)c2)n1]

more particularly
(+)-4-(4-Fluoro-2-methoxyphenyl)-N-{3-[(S-methylsulfonimidoyl)methyl]phenyl}-1,3,5-triazin-2-amine (compound A),
   acts in specific tumour types which had previously not yet been contemplated, viz. in lymphoma, preferably diffuse large B-cell lymphoma (DLBCL), mantle cell lymphoma, follicular lymphoma, diffuse large B-cell lymBurkitt's lymphoma, adult T-cell lymphoma (ATL) and Hodgkin's lymphoma, more preferably DLBCL and ATL.

4-(4-Fluoro-2-methoxyphenyl)-N-{3-[(S-methylsulfonimidoyl)methyl]phenyl}-1,3,5-triazin-2-amine (compound A) is a selected sulphoximine-substituted anilinopyrimidine derivative which can be separated into two stereoisomers, viz.:
(+)-4-(4-Fluoro-2-methoxyphenyl)-N-{3-[(S-methylsulfonimidoyl)methyl]phenyl}-1,3,5-triazin-2-amine (compound A) and
(−)-4-(4-Fluoro-2-methoxyphenyl)-N-{3-[(S-methylsulfonimidoyl)methyl]phenyl}-1,3,5-triazin-2-amine (compound A″).

Compound A′ is preferred and is in clinical development as Compound A′.

Where compound A is mentioned below, both the pure stereoisomers A′ and A″, and also any mixture of these two, are meant thereby.

The present invention is directed to the use of
4-(4-Fluoro-2-methoxyphenyl)-N-{3-[(S-methylsulfonimidoyl)methyl]phenyl}-1,3,5-triazin-2-amine (compound A) or one of its physiologically acceptable salts or enantiomers,
   more particularly
(+)-4-(4-Fluoro-2-methoxyphenyl)-N-{3-[(S-methylsulfonimidoyl)methyl]phenyl}-1,3,5-triazin-2-amine (compound A) or one of its physiologically acceptable salts,
   for the treatment and/or prophylaxis of lymphoma, preferably diffuse large B-cell lymphoma (DLBCL), mantle cell lymphoma, follicular lymphoma, diffuse large B-cell lymBurkitt's lymphoma, adult T-cell lymphoma (ATL) and Hodgkin's lymphoma, more preferably DLBCL and ATL.

The present application is further directed to the use of
4-(4-Fluoro-2-methoxyphenyl)-N-{3-[(S-methylsulfonimidoyl)methyl]phenyl}-1,3,5-triazin-2-amine or one of its physiologically acceptable salts or enantiomers,
   more particularly
(+)-4-(4-Fluoro-2-methoxyphenyl)-N-{3-[(S-methylsulfonimidoyl)methyl]phenyl}-1,3,5-triazin-2-amine or one of its physiologically acceptable salts,
   for preparing a medicament for treating lymphoma, preferably diffuse large B-cell lymphoma (DLBCL), mantle cell lymphoma, follicular lymphoma, diffuse large B-cell lymBurkitt's lymphoma, adult T-cell lymphoma (ATL) and Hodgkin's lymphoma, more preferably DLBCL and ATL.

Another aspect of the present invention is the

use of 4-(4-Fluoro-2-methoxyphenyl)-N-{3-[(S-methylsulfonimidoyl)methyl]phenyl}-1,3,5-triazin-2-amine (compound A) according to formula (I) or one of its physiologically acceptable salts or enantiomers

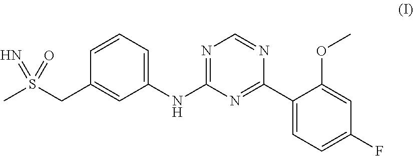 [SMILES: COc1cc(F)ccc1-c1ncnc(Nc2cccc(CS(C)(=N)=O)c2)n1]

more particularly
(+)-4-(4-Fluoro-2-methoxyphenyl)-N-{3-[(S-methylsulfonimidoyl)methyl]phenyl}-1,3,5-triazin-2-amine or one of its physiologically acceptable salts
   in the manufacture of a medicament for treating cancer in a subject, wherein the medicament is manufactured for treating lymphoma, preferably diffuse large B-cell lymphoma (DLBCL), mantle cell lymphoma, follicular lymphoma, diffuse large B-cell lymBurkitt's lymphoma, adult T-cell lymphoma (ATL) and Hodgkin's lymphoma, more preferably DLBCL and ATL.

The present application further provides
4-(4-Fluoro-2-methoxyphenyl)-N-{3-[(S-methylsulfonimidoyl)methyl]phenyl}-1,3,5-triazin-2-amine of formula I (compound A) or one of its physiologically acceptable salts or enantiomers

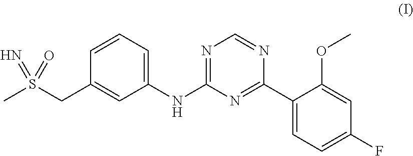 [SMILES: COc1cc(F)ccc1-c1ncnc(Nc2cccc(CS(C)(=N)=O)c2)n1]

more particularly
(+)-4-(4-Fluoro-2-methoxyphenyl)-N-{3-[(S-methylsulfonimidoyl)methyl]phenyl}-1,3,5-triazin-2-amine or one of its physiologically acceptable salts,
   for the use of treating lymphoma, preferably diffuse large B-cell lymphoma (DLBCL), mantle cell lymphoma, follicular lymphoma, diffuse large B-cell lymBurkitt's lymphoma, adult T-cell lymphoma (ATL) and Hodgkin's lymphoma, more preferably DLBCL and ATL.

The present invention is also directed to
4-(4-Fluoro-2-methoxyphenyl)-N-{3-[(S-methylsulfonimidoyl)methyl]phenyl}-1,3,5-triazin-2-amine of formula I (compound A) or one of its physiologically acceptable salts or enantiomers

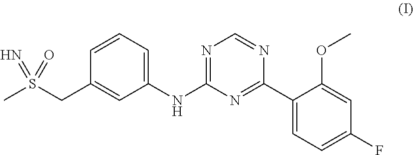 [SMILES: COc1cc(F)ccc1-c1ncnc(Nc2cccc(CS(C)(=N)=O)c2)n1]

more particularly
(+)-4-(4-Fluoro-2-methoxyphenyl)-N-{3-[(S-methylsulfonimidoyl)methyl]phenyl}-1,3,5-triazin-2-amine or one of its physiologically acceptable salts
   for the use in a method of treatment and/or prophylaxis of lymphoma, preferably diffuse large B-cell lymphoma (DLBCL), mantle cell lymphoma, follicular lymphoma, diffuse large B-cell lymBurkitt's lymphoma, adult T-cell lymphoma (ATL) and Hodgkin's lymphoma, more preferably DLBCL and ATL.

Another aspect of the present invention is a method of treatment and/or prophylaxis of lymphoma, preferably diffuse large B-cell lymphoma (DLBCL), mantle cell lymphoma, follicular lymphoma, diffuse large B-cell lymBurkitt's lymphoma, adult T-cell lymphoma (ATL) and Hodgkin's lymphoma, more preferably DLBCL and ATL using an effective amount of 4-(4-Fluoro-2-methoxyphenyl)-N-{3-[(S-methylsulfonimidoyl)methyl]phenyl}-1,3,5-triazin-2-amine (compound A) of formula I or one of its physiologically acceptable salts or enantiomers

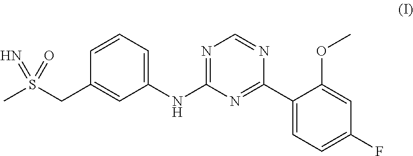 [SMILES: COc1cc(F)ccc1-c1ncnc(Nc2cccc(CS(C)(=N)=O)c2)n1]

more particularly
(+)-4-(4-Fluoro-2-methoxyphenyl)-N-{3-[(S-methylsulfonimidoyl)methyl]phenyl}-1,3,5-triazin-2-amine (compound A) or one of its physiologically acceptable salts.

The present application further provides pharmaceutical compositions comprising
4-(4-Fluoro-2-methoxyphenyl)-N-{3-[(S-methylsulfonimidoyl)methyl]phenyl}-1,3,5-triazin-2-amine or one of its physiologically acceptable salts or enantiomers,
   more particularly
(+)-4-(4-Fluoro-2-methoxyphenyl)-N-{3-[(S-methylsulfonimidoyl)methyl]phenyl}-1,3,5-triazin-2-amine or one of its physiologically acceptable salts,
   for treating lymphoma, preferably diffuse large B-cell lymphoma (DLBCL), mantle cell lymphoma, follicular lymphoma, diffuse large B-cell lymBurkitt's lymphoma, adult T-cell lymphoma (ATL) and Hodgkin's lymphoma, more preferably DLBCL and ATL.

The present invention is also directed to pharmaceutical compositions comprising
4-(4-Fluoro-2-methoxyphenyl)-N-{3-[(S-methylsulfonimidoyl)methyl]phenyl}-1,3,5-triazin-2-amine (compound A) of formula I or one of its physiologically acceptable salts or enantiomers

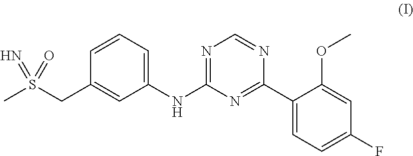 [SMILES: COc1cc(F)ccc1-c1ncnc(Nc2cccc(CS(C)(=N)=O)c2)n1]

more particularly
(+)-4-(4-Fluoro-2-methoxyphenyl)-N-{3-[(S-methylsulfonimidoyl)methyl]phenyl}-1,3,5-triazin-2-amine (compound A′ or one of its physiologically acceptable salts
   and at least one inert, nontoxic, pharmaceutically suitable adjuvant for the treatment and/or prophylaxis of lymphoma, preferably diffuse large B-cell lymphoma (DLBCL), mantle cell lymphoma, follicular lymphoma, diffuse large B-cell lymBurkitt's lymphoma, adult T-cell lymphoma (ATL) and Hodgkin's lymphoma, more preferably DLBCL and ATL.

The present application further provides combinations of
4-(4-Fluoro-2-methoxyphenyl)-N-{3-[(S-methylsulfonimidoyl)methyl]phenyl}-1,3,5-triazin-2-amine (compound A) or one of its physiologically acceptable salts or enantiomers,
   more particularly
(+)-4-(4-Fluoro-2-methoxyphenyl)-N-{3-[(S-methylsulfonimidoyl)methyl]phenyl}-1,3,5-triazin-2-amine (compound A′ or one of its physiologically acceptable salts,
   with at least one further active ingredient for treating lymphoma, preferably diffuse large B-cell lymphoma (DLBCL), mantle cell lymphoma, follicular lymphoma, diffuse large B-cell lymBurkitt's lymphoma, adult T-cell lymphoma (ATL) and Hodgkin's lymphoma, more preferably DLBCL and ATL

The present invention is also directed to pharmaceutical combinations comprising
4-(4-Fluoro-2-methoxyphenyl)-N-{3-[(S-methylsulfonimidoyl)methyl]phenyl}-1,3,5-triazin-2-amine (compound A) of formula I or one of its physiologically acceptable salts or enantiomers

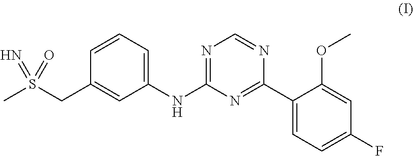 [SMILES: COc1cc(F)ccc1-c1ncnc(Nc2cccc(CS(C)(=N)=O)c2)n1]

more particularly
(+)-4-(4-Fluoro-2-methoxyphenyl)-N-{3-[(S-methylsulfonimidoyl)methyl]phenyl}-1,3,5-triazin-2-amine (compound A′ or one of its physiologically acceptable salts,
   and at least one or more further active ingredients for the treatment and/or prophylaxis of lymphoma, preferably diffuse large B-cell lymphoma (DLBCL), mantle cell lymphoma, follicular lymphoma, diffuse large B-cell lymBurkitt's lymphoma, adult T-cell lymphoma (ATL) and Hodgkin's lymphoma, more preferably DLBCL and ATL.

The use of the physiologically tolerable salts of compound A should likewise be considered to be covered by the present invention.

Physiologically safe salts of compound A encompass acid addition salts of mineral acids, carboxylic acids and sulphonic acids, for example salts of hydrochloric acid, hydrobromic acid, sulphuric acid, phosphoric acid, methanesulphonic acid, ethanesulphonic acid, toluenesulphonic acid, benzenesulphonic acid, naphthalenedisulphonic acid, acetic acid, trifluoroacetic acid, propionic acid, lactic acid, tartaric acid, malic acid, citric acid, fumaric acid, maleic acid and benzoic acid.

Physiologically safe salts of compound A also encompass salts of customary bases, such as, by way of example and preferably, alkali metal salts (e.g. sodium and potassium salts), alkaline earth metal salts (e.g. calcium and magnesium salts) and ammonium salts derived from ammonia or organic amines having from 1 to 16 C atoms, such as, by way of example and preferably, ethylamine, diethylamine, triethylamine, ethyldiisopropylamine, monoethanolamine, diethanolamine, triethanolamine, dicyclohexylamine, dimethylaminoethanol, procaine, dibenzylamine, N-methylmorpholine, arginine, lysine, ethylenediamine and N-methylpiperidine.

The present invention further provides drugs containing compound A and at least one or more further active ingredients for treating lymphoma, preferably diffuse large B-cell lymphoma (DLBCL), mantle cell lymphoma, follicular lymphoma, diffuse large B-cell lymBurkitt's lymphoma, adult T-cell lymphoma (ATL) and Hodgkin's lymphoma, more preferably DLBCL and ATL.

Compound A may have systemic and/or local activity. For this purpose, it can be administered in a suitable manner, such as, for example, orally, parenterally, via the pulmonary route, nasal, sublingually, lingually, buccally, rectally, vaginally, dermally, transdermally, conjuntivally, otically or as an implant or stent.

For these administration routes, compound A according to the invention may be administered in suitable administration forms.

Suitable for oral administration forms which function according to the prior art and deliver compound A of the invention rapidly and/or in a modified manner and which comprise compound A according to the invention in crystalline and/or amorphised and/or dissolved form, such as, for example, tablets (uncoated or coated tablets, for example with coatings which are resistant to gastric juice or dissolve with a delay or are insoluble and control the release of the compound of the invention), tablets which disintegrate rapidly in the oral cavity, or films/wafers, films/lyophilisates, capsules (for example hard or soft gelatine capsules), sugar-coated tablets, granules, pellets, powders, emulsions, suspensions, aerosols or solutions.

Parenteral administration can be effected with avoidance of an absorption step (for example intravenous, intraarterial, intracardial, intraspinal or intralumbal) or with inclusion of absorption (for example intramuscular, subcutaneous, intracutaneous, percutaneous or intraperidoneal). Administration forms which are suitable for parenteral administration are, inter alia, preparations for injection and infusion in the form of solutions, suspensions, emulsions, lyophilisates or sterile powders.

Examples which are suitable for other administration routes are pharmaceutical forms for inhalation [inter alia power inhalers, nebulisers], nasal drops, solutions, sprays; tablets, films/wafers or capsules, to be administered lingually, sublingually or buccaly, suppositories, preparations for the eyes and the ears, eye baths, ocular insert, ear drops, ear powders, ear-rinses, ear tampons, vaginal capsules, aqueous suspensions (lotions, mixturae agitandae), lipophilic suspensions, ointments, creams, transdermal therapeutic systems (such as, for example, patches), milk, pastes, foams, dusting powders, implants or stents.

Compound A can be converted into the stated administration forms. This can be affected in a manner known per se by mixing with inert, non-toxic, pharmaceutically suitable adjuvants. These adjuvants include, inter alia,
fillers and excipients (for example cellulose, microcrystalline cellulose, such as, for example, Avicel®, lactose, mannitol, starch, calcium phosphate such as, for example, Di-Cafos®),
ointment bases (for example petroleum jelly, paraffins, triglycerides, waxes, wool wax, wool wax alcohols, lanolin, hydrophilic ointment, polyethylene glycols),
bases for suppositories (for example polyethylene glycols, cacao butter, hard fat)
solvents (for example water, ethanol, Isopropanol, glycerol, propylene glycol, medium chain-length triglycerides fatty oils, liquid polyethylene glycols, paraffins),
surfactants, emulsifiers, dispersants or wetters (for example sodium dodecyle sulphate, lecithin, phospholipids, fatty alcohols such as, for example, Lanette®, sorbitan fatty acid esters such as, for example, Span®, polyoxyethylene sorbitan fatty acid esters such as, for example, Tween®, polyoxyethylene fatty acid glycerides such as, for example, Cremophorr, polyoxethylene fatty acid esters, polyoxyethylene fatty alcohol ethers, glycerol fatty acid esters, poloxamers such as, for example, Pluronic®),
buffers and also acids and bases (for example phosphates, carbonates, citric acid, acetic acid, hydrochloric acid, sodium hydroxide solution, ammonium carbonate, trometamol, triethanolamine)
isotonicity agents (for example glucose, sodium chloride),
adsorbents (for example highly-disperse silicas)
viscosity-increasing agents, gel formers, thickeners and/or binders (for example polyvinylpyrrolidon, methylcellulose, hydroxypropylmethylcellulose, hydroxypropylcellulose, carboxymethylcellulose-sodium, starch, carbomers, polyacrylic acids such as, for example, Carbopol®, alginates, gelatine),
disintegrants (for example modified starch, carboxymethylcellulose-sodium, sodium starch glycolate such as, for example, Explotab®, cross-linked polyvinylpyrrolidon, croscarmellose-sodium such as, for example, AcDiSol®),
flow regulators, lubricants, glidant and mould release agents (for example magnesium stearate, stearic acid, talc, highly-disperse silicas such as, for example, Aerosil®),
coating materials (for example sugar, shellac) and film formers for films or diffusion membranes which dissolve rapidly or in a modified manner (for example polyvinylpyrrolidones such as, for example, Kollidon®, polyvinyl alcohol, hydroxypropylmethylcellulose, hydroxypropylcellulose, ethylcellulose, hydroxypropylmethylcellulose phthalate, cellulose acetate, cellulose acetate phthalate, polyacrylates, polymethacrylates such as, for example, Eudragit®),
capsule materials (for example gelatine, hydroxypropylmethylcellulose),
synthetic polymers (for example polylactides, polyglycolides, polyacrylates, polymethacrylates such as, for example, Eudragit®, polyvinylpyrrolidones such as, for example, Kollidon®, polyvinyl alcohols, polyvinyl acetates, polyethylene oxides, polyethylene glycols and their copolymers and blockcopolymers),
plasticisers (for example polyethylene glycols, propylene glycol, glycerol, triacetine, triacetyl citrate, dibutyl phthalate),
penetration enhancers,
stabilisers (for example antioxidants such as, for example, ascorbic acid, ascorbyl palmitate, sodium ascorbate, butylhydroxyanisole, butylhydroxytoluene, propyl gallate),
preservatives (for example parabens, sorbic acid, thiomersal, benzalkonium chloride, chlorhexidine acetate, sodium benzoate),
colourants (for example inorganic pigments such as, for example, iron oxides, titanium dioxide),
flavourings, sweeteners, flavour- and/or odour-masking agents.

The present invention furthermore relates to medicaments which comprise at least one compound according to the invention, conventionally together with one or more inert, non-toxic, pharmaceutically suitable adjuvants, and to their use for the above mentioned purposes.

### Dosage and Treatment Regimen

The dosage and the treatment regimen can and must be varied depending on the carcinoma type and the treatment goal.

The daily dose is generally between 20 mg and 850 mg and can be divided into a plurality of identical or different dosage units, preferably 2 which can be taken simultaneously or according to a certain time schedule.

In particular the daily dose is between 30 mg and 500 mg and can be divided into a plurality of identical or different dosage units, preferably 2 which can be taken simultaneously or according to a certain time schedule.

A preferred daily dose is between 20 mg and 400 mg and can be divided into a plurality of identical or different dosage units, preferably 2 which can be taken simultaneously or according to a certain time schedule.

More particularly, the daily dose is between 40 mg and 300 mg and can be divided into a plurality of identical or different dosage units, preferably 2 which can be taken simultaneously or according to a certain time schedule.

A more preferred daily dose is between 20 mg and 200 mg and can be divided into a plurality of identical or different dosage units, preferably 2 which can be taken simultaneously or according to a certain time schedule.

An even more preferred daily dose is between 50 mg and 180 mg and can be divided into a plurality of identical or different dosage units, preferably 2 which can be taken simultaneously or according to a certain time schedule.

This applies both to monotherapy and to combination therapy with other anti-hyperproliferative, cytostatic or cytotoxic substances, the combination therapy possibly requiring a reduction in dose.

The treatment can be carried out in regularly repeated cycles. Treatment cycles may have varying duration, such as 21 days or 28 days, whereby dosing is given continuously, or intermittently. Preferred is a cycle length of 28 days, whereby dosing is given continuously, or intermittently.

Continuous schedules involve daily dosing, for example, 21 daily doses in a 21-day cycle, or 28 daily doses in a 28-day cycle. A preferred continuous schedule is 28 daily doses in a 28 daily cycle.

Intermittent schedules involve a period of treatment followed by a period of non-treatment, for example in a cycle of 21 days, or a cycle of 28 days. A preferred cycle duration for an intermittent schedule is 28 days.

The period of treatment may be repeated more than once in a given treatment cycle.

The period of treatment may be for example 1 to 21 days, more preferably 3 to 14 days.

An even more preferred intermittent schedule involves treatment for 3 days followed by non-treatment for 4 days, repeated every week in such a way that a 28-day treatment cycle is completed.

Treatment is successful when there is at least disease stabilisation and the adverse effects occur to an extent which is easily treatable, but at least easily acceptable. Thus the number of cycles of treatment applied may vary from patient to patient, according to treatment response and tolerability.

Treatment is successful when there is at least disease stabilisation and the adverse effects occur to an extent which is easily treatable, but at least easily acceptable.

Compound A can be used on its own or, if required, in combination with one or more other pharmacologically effective substances, provided said combination does not lead to undesired and unacceptable adverse effects. The present invention therefore further provides drugs containing compound A according to the invention and one or more further active ingredients, in particular for treating and/or preventing the above-mentioned diseases.

For example, compound A can be combined with known anti-hyperproliferative, cytostatic or cytotoxic substances for treating cancers. The combination compound A according to the invention with other substances in use for cancer therapy or else with radiotherapy is especially advisable.

Examples of suitable active ingredients for combination purposes include:

abraxane, afinitor, aldesleukin, alendronic acid, alfaferone, alitretinoin, allopurinol, aloprim, aloxi, altretamine, aminoglutethimide, amifostine, amrubicin, amsacrine, anastrozole, anzemet, aranesp, arglabin, arsenic trioxide, aromasin, 5-azacytidine, azathioprine, BCG or tice-BCG, bestatin, betamethasone acetate, betamethasone sodium phosphate, bexarotene, bleomycin sulphate, broxuridine, bortezomib, busulfan, calcitonin, campath, capecitabine, carboplatin, casodex, cefesone, celmoleukin, cerubidine, chlorambucil, cisplatin, cladribine, clodronic acid, cyclophosphamide, cytarabine, dacarbazine, dactinomycin, daunoxome, decadron, decadron phosphate, delestrogen, denileukin diftitox, depo-medrol, deslorelin, dexrazoxane, diethylstilbestrol, diflucan, docetaxel, doxifluridine, doxorubicin, dronabinol, DW-166HC, eligard, elitek, ellence, emend, epirubicin, epoetin alfa, epogen, eptaplatin, ergamisol, estrace, estradiol, estramustine sodium phosphate, ethinyl estradiol, ethyol, etidronic acid, etopophos, etoposide, fadrozole, fareston, filgrastim, finasteride, fligrastim, floxuridine, fluconazole, fludarabine, 5-fluorodeoxyuridine monophosphate, 5-flurouracil (5-FU), fluoxymesterone, flutamide, formestane, fosteabine, fotemustine, fulvestrant, gammagard, gemcitabine, gemtuzumab, gleevec, gliadel, goserelin, granisetron hydrochloride, histrelin, hycamtin, hydrocortone, erythro-hydroxynonyladenine, hydroxyurea, ibritumomab tiuxetan, idarubicin, ifosfamide, interferon alpha, interferon alpha 2, interferon alpha 2α, interferon alpha 2β, interferon alpha n1, interferon alpha n3, interferon beta, interferon gamma 1α, interleukin 2, intron A, iressa, irinotecan, kytril, lapatinib, lentinan sulphate, letrozole, leucovorin, leuprolide, leuprolide acetate, levamisole, levofolinic acid calcium salt, levothroid, levoxyl, lomustine, lonidamine, marinol, mechlorethamine, mecobalamin, medroxyprogesterone acetate, megestrol acetate, melphalan, menest, 6-mercaptopurine, mesna, methotrexate, metvix, miltefosine, minocycline, mitomycin C, mitotane, mitoxantrone, modrenal, myocet, nedaplatin, neulasta, neumega, neupogen, nilutamide, nolvadex, NSC-631570, OCT-43, octreotide, ondansetron hydrochloride, orapred, oxaliplatin, paclitaxel, pcdiapred, pegaspargase, pegasys, pentostatin, picibanil, pilocarpine hydrochloride, pirarubicin, plicamycin, porfimer sodium, prednimustine, prednisolone, prednisone, premarin, procarbazine, procrit, raltitrexed, RDEA119, rebif, rhenium-186 etidronate, rituximab, roferon-A, romurtide, salagen, sandostatin, sargramostim, semustine, sizofiran, sobuzoxane, solu-medrol, streptozocin, strontium-89 chloride, synthroid, tamoxifen, tamsulosin, tasonermin, tastolactone, taxotere, teceleukin, temozolomide, teniposide, testosterone propionate, testred, thioguanine, thiotepa, thyrotropin, tiludronic acid, topotecan, toremifene, tositumomab, trastuzumab, treosulfan, tretinoin, trexall, trimethylmelamine, trimetrexate, triptorelin acetate, triptorelin pamoate, UFT, uridine, valrubicin, vesnarinone, vinblastine, vincristine, vindesine, vinorelbine, virulizin, zinecard, zinostatin stimalamer, zofran; ABI-007, acolbifene, actimmune, affinitak, aminopterin, arzoxifene, asoprisnil, atamestane, atrasentan, BAY 43-9006 (sorafenib), avastin, CCI-779, CDC-501, celebrex, cetuximab, crisnatol, cyproterone acetate, decitabine, DN-101, doxorubicin MTC, dSLIM, dutasteride, edotecarin, eflornithine, exatecan, fenretinide, histamine dihydrochloride, histrelin hydrogel implant, holmium-166 DOTMP, ibandronic acid, interferon gamma, intron-PEG, ixabepilone, keyhole limpet hemocyanin, L-651582, lanreotide, lasofoxifene, libra, lonafamib, miproxifen, minodronate, MS-209, liposomal MTP-PE, MX-6, nafarelin, nemorubicin, neovastat, nolatrexed, oblimersen, onco-TCS, osidem, paclitaxel polyglutamate, pamidronate disodium, PN-401, QS-21, quazepam. R-1549, raloxifene, ranpirnase, 13-cis-retinoic acid, satraplalin, seocalcitol, T-138067, tarceva, taxoprexin, thymosin alpha 1, tiazofurin, tipifarnib, tirapazamine, TLK-286, toremifene, transMID-107R, valspodar, vapreotide, vatalanib, verteporfin, vinflunine, Z-100, zoledronic acid, and also combinations thereof.

In a preferred embodiment, compound A of the present invention can be combined with the following active ingredients:

131I-chTNT, abarelix, abiraterone, aclarubicin, aldesleukin, alemtuzunab, alitretinoin, altretamine, aminoglutethimide, amrubicin, amsacrine, anastrozole, arglabin, arsenic trioxide, asparaginase, azacitidine, basiliximab, BAY 80-6946, belotecan, bendamustine, bevacizumab, bexarotene, bicalutamide, bisantrene, bleomycin, bortezomib, buserelin, busulfan, cabazitaxel, calcium folinate, calcium levofolinate, capecitabine, carboplatin, carmofur, carmustine, catumaxomab, celecoxib, celmoleukin, ceruximab, chlorambucil, chlormadinone, chlormethine, cisplatin, cladribine, clodronic acid, clofarabine, crisantaspase, cyclophosphamide, cyproterone, cytarabine, dacarbazine, dactinomycin, darbepoetin alfa, dasatinib, daunorubicin, decitabine, degarelix, denileukin diftitox, denosumab, deslorelin, dibrospidium chloride, docetaxel, doxifluridine, doxorubicin, doxorubicin+estrone, eculizumab, edrecolomab, elliptinium acetate, eltrombopag, endostatin, enocitabine, epirubicin, epitiostanol, epoetin alfa, epoetin beta, eptaplatin, eribulin, erlotinib, estradiol, estramustine, etoposide, everolimus, exemestane, fadrozole, filgrastim, fludarabine, fluorouracil, flutamide, formestane, fotemustine, fulvestrant, gallium nitrate, ganirelix, gefitinib, gemcitabine, gemtuzumab, glutoxim, goserelin, histamine dihydrochloride, histrelin, bydroxycarbamide, I-125 seeds, ibandronic acid, ibritumomab tiuxetan, idarubicin, ifosfamide, imatinib, imiquimod, improsulfan, interferon alpha, interferon beta, interferon gamma, ipilimumab, irinotecan, ixabepilone, lanreotide, lapatinib, lenalidomide, lenograstim, lentinan, letrozole, leuprorelin, levamisole, lisuride, lobaplatin, lomustine, lonidamine, masoprocol, medroxyprogesterone, megestrol, melphalan, mepitiostane, mercaptopurine, methotrexate, methoxsalen, methyl aminolevulinate, methyltestosterone, mifamurtide, miltefosine, miriplatin, mitobronitol, mitoguazone, mitolactol, mitomycin, mitotane, mitoxantrone, nedaplatin, nelarabine, nilotinib, nilutamide, nimotuzumab, nimustine, nitracrine, ofatumumab, omeprazole, oprelvekin, oxaliplatin, p53 gene therapy, paclitaxel, palifermin, palladium-103 seed, pamidronic acid, panitumumab, pazopanib, pegaspargase, PEG-epoetin beta (methoxy-PEG-epoetin beta), pegfilgrastim, peginterferon alfa 2b, pemetrexed, pentazocine, pentostatin, peplomycin, perfosfamide, picibanil, pirarubicin, plerixafor, plicamycin, poliglusam, polyestradiol phosphate, polysaccharide-K, porfimer sodium, pralatrexate, prednimustine, procarbazine, quinagolide, radium-223 chloride, raloxifene, raltitrexed, ranimustine, razoxane, refametinib, regorafenib, risedronic acid, rituximab, romidepsin, romiplostim, sargramostim, sipuleucel-T, sizofiran, sobuzoxane, sodium glycididazole, sorafenib, streptozocin, sunitinib, talaporfin, ramibarotene, tamoxifen, tasonermin, teceleukin, tegafur, tegafur+gimeracil+oteracil, temoporfin, temozolomide, temsirolimus, teniposide, testosterone, tetrofosmin, thalidomide, thiotepa, thymalfasin, tioguanine, tocilizumab, topotecan, toremifene, tositumomab, trabectedin, trastuzumab, treosulfan, tretinoin, trilostane, triptorelin, trofosfamide, tryptophan, ubenimex, valrubicin, vandetanib, vapreotide, vemurafenib, vinblastine, vincristine, vindesine, vinflunine, vinorelbine, vorinostat, vorozole, yttrium-90 glass microspheres, zinostatin, zinostatin stimalamer, zoledronic acid, zorubicin.

Promisingly, compound A can also be combined with biological therapeutics such as antibodies (e.g. avastin, rituxan, erbitux, herceptin, cetuximab) and recombinant proteins.

Compound A can also achieve positive effects in combination with other therapies directed against angiogenesis, such as, for example, with avastin, axitinib, regorafenib, recentin, sorafenib or sunitinib. Combinations with inhibitors of the proteasome and of mTOR and also antihormones and steroidal metabolic enzyme inhibitors are especially useful because of their favourable profile of adverse effects.

In general, the combination of compound A with other cytostatic or cytotoxic agents makes it possible to pursue the following goals:
improved efficacy in slowing the growth of a tumour, in reducing its size or even in completely eliminating it in comparison with treatment using an individual active ingredient;
the possibility of employing the chemotherapeutics used in a lower dosage than in the case of monotherapy;
the possibility of a more tolerable therapy with fewer adverse effects in comparison with individual administration;
the possibility of treating a broader spectrum of tumour diseases;
achieving a higher response rate to the therapy;
longer patient survival time in comparison with current standard therapy.

Furthermore, compound A according to the invention can also be used in connection with radiotherapy and/or a surgical intervention.

### EXAMPLES

### 1. Preparation of Compound A′

Compound A′ was prepared according to the procedure described in example 4 of WO2012/160034.

### 2. In-Vitro Experiments

### 2.1. Methods

### 2.1.1 Cell Lines

TABLE 1
List of the cell lines investigated.

| | Example |
|---|---|
| Tumour indication | Cell line |
| DLBCL, ABC subtype | HBL1 |
| DLBCL, ABC subtype | OCI-LY-3 |
| DLBCL, GCB subtype | DB |
| DLBCL, GCB subtype | SU-DHL-6 |
| DLBCL, GCB subtype | HT |
| DLBCL, GCB subtype | OCI-LY-19 |
| DLBCL, GCB subtype | SU-DHL-8 |
| DLBCL, GCB subtype | SU-DHL-10 |
| DLBCL, GCB subtype | SU-DHL-4 |
| Mantle cell lymphoma | Jeko-1 |
| Follicular lymphoma | Karpas422 |
| Burkitt's lymphoma | Ramos |
| Hodgkin lymphoma | KM-H2 |
| ATL | ATN-1 |
| ATL | MJ |
| ATL | MT-1 |
| ATL | TL-Om1 |
| ATL | S-YU |
| HTLV-1-immortalized cell lines | MT-2 |
| HTLV-1-immortalized cell lines | MT-4 |
| HTLV-1-immortalized cell lines | TL-SU |

ATN-1, MJ, MT-1, TL-Om1, and S-YU are ATL cell lines, whereas MT-2, MT-4, and TL-Su are HTLV-1-immortalized cell lines, as previously described (Clin Cancer Res. 2003; 9(10):3625-3634.; Cancer Sci. 2012; 103 (10):1764-1773; Jpn J Cancer Res. 1996; 87(9):887-892.; Science. 1983; 219(4586):856-859)).

### 2.1.2 Cell Proliferation Assay

The proliferation of all lymphoma cell lines, other than S-YU, in the presence of different concentrations of Compound A′ for 72 h was assessed using CellTiter Glo kits (Promega Corporation, Madison, Wis.). Proliferation of S-YU in the presence of recombinant human interleukin-2 (IL-2) at a final concentration of 100 IU/mL, with different concentrations of Compound A′ for 24 h, was assessed in the same manner. All expressed values were averages of triplicate experiments, and IC50 was calculated using GraphPad Prism 5 (GraphPad Software, San Diego, Calif.) according to the manufacturer's instructions or the MTS software.

### 2.2 In-Vitro Results

Table 2 summarizes the results in the proliferation assays.

TABLE 2
List of the cell lines investigated and
results of the proliferation assays.

| | Example | Compound A\acute{ } |
|---|---|---|
| Tumour indication | Cell line | IC₅₀ [nmol/l] |
| DLBCL, ABC subtype | HBL1 | 540 |
| DLBCL, ABC subtype | OCI-LY-3 | 340 |
| DLBCL, GCB subtype | DB | 570 |
| DLBCL, GCB subtype | SU-DHL-6 | 360 |
| DLBCL, GCB subtype | HT | 710 |
| DLBCL, GCB subtype | OCI-LY-19 | 480 |
| DLBCL, GCB subtype | SU-DHL-8 | 610 |
| DLBCL, GCB subtype | SU-DHL-10 | 600 |
| DLBCL, GCB subtype | SU-DHL-4 | 170 |
| Mantle cell lymphoma | Jeko-1 | 740 |
| Follicular lymphoma | Karpas422 | 660 |
| Burkitt's lymphoma | Ramos | 1100 |
| Hodgkin lymphoma | KM-H2 | 2200 |
| ATL | ATN-1 | 440 |
| ATL | MJ | 540 |
| ATL | MT-1 | 1200 |
| ATL | TL-Om1 | 790 |
| ATL | S-YU* | 2960 |
| HTLV-1-immortalized cell lines | MT-2 | 520 |
| HTLV-1-immortalized cell lines | MT-4 | 1410 |
| HTLV-1-immortalized cell lines | TL-SU | 2130 |

*After 24 hours of incubation with the substance

These in vitro data indicate an efficient inhibition of the proliferation of different types of lymphoma cell by Compound A′. These data recommend Compound A′ for the treatment of patients with lymphomas, preferably with DLBCL and ATL.

### 3. In-Vive Experiments Diffuse Large B-Cell Lymphoma (DLBCL)

The aim of the present experiments was to assess the in vivo efficacy and tolerability of Compound A′ in monotherapy in the DLBCL OCI-LY-7 tumour model subcutaneously implanted in NOG mice.

### 3.1 Acronyms and Abbreviations

TABLE 3
Acronyms and abbreviations

| | |
|---|---|
| BW | Body weight |
| BW₀ | Individual body weight at day 0 |
| BWₓ | Individual body weight at day X |
| BWL | Body weight loss |
| DLBCL | Diffuse large B-cell lymphoma |
| ABC | Activated B-cell type |
| GBC | Germinal-centre B-cell type |
| i.v. | Intravenously |
| n/a | Not applicable |
| NOD/SCID | Non-obese diabetic/severe combined immunodeficiency |
| NOG mouse | NOD/Shi-scid/IL-2Rγ^{null} mouse |
| p.o. | Per os, orally |
| T/C | Treatment to control ratio |
| RTV | Relative tumour volume |

### 3.2 Design

In vivo efficacy was determined in female NOG mice bearing subcutaneous DLBCL OCI-LY-7 xenografts. Compound A′ was assessed at one dose level in monotherapy. Anti-tumour activity and tolerability of the treated group was assessed using the vehicle control group as a reference.

TABLE 4
Design in-vivo experiments

| Group | | Total Daily Dose | Schedule | Appl. | No. of |
|---|---|---|---|---|---|
| ID | Therapy | [mg/kg/day] | [Dosing days] | Route | Animals |
| 1 | Vehicle | 5 mL/kg/day | 0, 7, 14, 21 | i.v. | 10 |
| 2 | Com- | 25 | 0-27 | p.o. | 10 |
| | pound A\acute{ } | | | | |

### 3.3 Experimental Procedures

### 3.3.1. Specific Animal Information

Mouse strain, sex: NOG, female
Animals supplied by: Taconic, Denmark
Approximate age at implantation: 5-7 weeks

### Total Number of Mice

Efficacy test (implanted/randomised): 29/20

### Housing Conditions

The animals were housed in individually ventilated cages. The animals were monitored twice daily. All materials were autoclaved prior to use. Food and water were provided ad libitum.

### 3.3.2 Tumour Information

### 3.3.2.1 Characterisation of Test Tumours

The tumour model used in this study was derived from a commercially available DLBCL cell line OCI-LY7.

### 3.3.2.2 Tumour Implantation

DLBCL tumour fragments, derived from the OCI-LY-7 cell line, were obtained from xenografts in serial passage in nude mice and placed in PBS containing 10% penicillin/streptomycin. Tumour fragments (one fragment per animal; 3-4 mm edge length) were then subcutaneously implanted in the flank of NOG recipient mice under isofluorane anaesthesia.

### 3.3.3 Randomisation

Animals and tumour implants were monitored daily until the maximum number of implants showed clear signs of beginning solid tumour growth. At randomisation, the volume of growing tumours was initially determined. Animals bearing one tumour of a volume of 50-250 mm³, preferably 80-200 mm³, were distributed in experimental groups according to the study protocol, considering a comparable median and mean of group tumour volume of approximately 100-120 mm³. The result of the randomisation was documented and maintained with the experimental data. Animals not randomised were euthanised. The day of randomisation is designated as day 0 of an experiment.

### 3.3.4. Test Reagents

Vehicle: 80% (m/V) PEC400 in water for injection
Compound A′: preparation of a dosing solution (2.5 mg/ml) once weekly by diluting the Compound A′ powder at 0.25% (w/v) in vehicle; storage of the dosing solution at 4° C.; dosing volume 10 ml/kg

### 3.3.5. Observations and Calculations

### 3.3.5.1 Mortality

Mortality checks were conducted daily during routine monitoring.

### 3.3.5.2 Body Weight

Mice were weighed twice a week. Relative body weights of individual mice in % were calculated by dividing the individual body weight on day X (BW_{X}) by the individual body weight on day 0 (BW₀) multiplied by 100 according to the formula:

$$Relative Body Weight ( {Day}_{x} ) [ \% ] = \frac{{BW}_{x}}{{BW}_{0}} \times 100$$

Group median relative body weights were calculated as well, considering only the weights of mice that were alive on the day in question.

### 3.3.5.3 Tumour Volume

The tumour volumes were determined by two-dimensional measurement with a caliper on the day of randomisation (day 0) and then twice weekly (i.e. on the same days on which mice were weighed). Tumour volumes were calculated according to the formulas:

Tumour volume=(a×b²)×0.5

where a represents the largest and b the perpendicular tumour diameter.

Relative volumes of individual tumours (RTVs) for Day x were calculated by dividing the absolute individual tumour volume on Day x (Tₓ) by the absolute individual tumour volume of the same tumour on Day 0 (T₀) multiplied by 100%:

RTVₓ [%]=(Tₓ/T₀)×100

### 3.3.5.4 Anti-Tumour Activity

Anti-tumour activity was evaluated as maximum tumour volume inhibition versus the vehicle control group.

### 3.3.5.5 Tumour Inhibition, Test/Control Value in %

Tumour inhibition for a particular day (T/C in %) was calculated from the ratio of the median RTV values of test versus control groups multiplied by 100.

$$T / C ( {Day}_{x} ) [ \% ] = \frac{Median relative tumour volume of the test group on {Day}_{x}}{\begin{matrix}Median relative tumour \\ volume of the control group on {Day}_{x}\end{matrix}} = 100$$

The minimum (or optimum) T/C % value recorded for a particular test group during an experiment represents the maximum anti-tumour activity for the respective treatment. T/C values were calculated if at least 50% of the randomised animals in a group were alive on the day in question.

### 3.3.5.6 Efficacy Criteria

Group optimum T/C values (in %) were used for activity rating as follows:

TABLE 5
Efficacy criteria.

| | | |
|---|---|---|
| − | Inactive | T/C ≧ 65% |
| +/− | Borderline activity | 50% ≦ T/C < 65% |
| + | Moderate activity | 25% ≦ T/C < 50% |
| ++ | High activity | 10% ≦ T/C < 25% |
| +++ | Very high activity | 5% ≦ T/C < 10% |
| ++++ | Complete remission | T/C < 5% |

### 3.4 Results

### 3.4.1 Anti-Tumour Efficacy of Compound A′ in Xenograft-Bearing Mice

Compound A′ was assessed at one dose level in the DLBCL OCI-LY-7 tumour model subcutaneously implanted into NOG mice.

Excellent anti-tumour activity, classed as complete remission, was observed with Compound A′ treatment in the OCI-LY-7 tumour model with a minimum T/C value of 2.6%.

OCI-LY-7 tumour growth was significantly reduced by Compound A′ treatment as compared to the respective vehicle control group and determined by the non-parametric Mann-Whitney-Wilcoxon U-test.

TABLE 6
Summary of Anti-tumour Efficacy of Compound A\grave{ }.

| | | | | | |
|---|---|---|---|---|---|
| Group | | Dose Level | | Min. T/C | Activity |
| ID | Therapy | [mg/kg/day] | Schedule | [%] (Day) | Rating |

OCI-LY-7

| | | | | | |
|---|---|---|---|---|---|
| 1 | Vehicle | 5 ml/kg/day | 0, 7, 14, 21 | n/a | n/a |
| 2 | Com- | 25 | 0-27 | 2.6 (19) | ++++ |
| | pound A\acute{ } | | | | |

* Vehicle, 80% PEG400 in water for injection

In conclusion, these data indicate significant and meaningful anti-tumour activity of Compound A′ in patients with diffuse large B-cell lymphoma (DLBCL).

### 3.4.2. Survival and Body Weight Changes

No median BWL was observed in animals receiving vehicle control, while moderate median BWL of 17.9% was recorded for Compound A′-treated animals. Survival rates for vehicle and Compound A′ groups were 100% and 80%, respectively.

In conclusion, Compound A′ showed an acceptable tolerability profile in lymphoma xenograft bearing mice.

### 3.5. Summary and Conclusion

The in vivo efficacy and tolerability of Bayer Healthcare's investigational compound Compound A′ was assessed in monotherapy in the DLBCL OCI-LY-7 xenograft model subcutaneously implanted into female NOG mice. Compound A′ was administered orally at one dose level (25 mg/kg/day), once daily, in monotherapy and treatment was initiated once subcutaneous tumours were established. A vehicle-treated control group was included in each experiment. Group sizes were ten mice per group. Anti-tumour activity (tumour growth inhibition) and tolerability of Compound A′ was assessed using the vehicle control group as a reference.

Excellent anti-tumour activity was observed in the OCI-LY-7 tumour model with a minimum T/C value of 2.6%.

OCI-LY-7 tumour growth was significantly reduced by Compound A′ treatment as compared to the respective vehicle control group (determined by the non-parametric Mann-Whitney-Wilcoxon U-test). No median BWL was observed in animals receiving vehicle control, while moderate to median BWL of 17.9% was recorded for Compound A′-treated animals. Survival rates for vehicle and Compound A′ groups were 100% and 80%, respectively.

These data indicate a significant and meaningful anti-tumour activity of Compound A′ in patients with diffuse large B-cell lymphoma (DLBCL).

### 4. In-Vivo Experiments Adult T-Cell Lymphoma (ATL)

### 4.1 Animals

NOD/Shi-scid, IL-2Rγ^{null} (NOG) mice were purchased from the Central Institute for Experimental Animals (Kanagawa, Japan) and used at between 6-8 weeks of age.

4.2 ATL Cell-Bearing Mice Treated with Compound A′

A leukemic cell clone from a patient with ATL, which could be serially transplanted into SCID mice and was designated S-YU as reported previously (Eur J Haematol. 2014; 92(3):219-228), was injected intraperitoneally (i.p.) into NOG mice. Three to four weeks after i.p. injection, NOG mice developed intraperitoneal masses within the mesentery. Cells from these intraperitoneal masses were suspended in RPMI-1640 and inoculated i.p. into healthy NOG mice, which then presented with disease features identical to those of the original mice. ATL tumor cells (S-YU) from the intraperitoneal masses were suspended in RPMI-1640, and 1.0×10⁷ cells were inoculated i.p. into each of 16 naive NOG mice. The animals were randomly divided into two groups of eight each for treatment with Compound A′ or vehicle, seven days after ATL cell inoculations. Compound A′ was formulated in 40% PEG400 in water at a final concentration of 2.5 mg/mL. Mice were treated by oral application of 12.5 mg/kg Compound A′ (0.25 mg/100 μL per mouse) or vehicle (100 μL), once daily for 18 days (7-24 days after ATL cell inoculations). Therapeutic efficacy was then evaluated 25 days after ATL cell inoculations.

ATL cells from intraperitoneal masses suspended in RPMI-1640 were also inoculated i.p. into another 14 naive NOG mice at 1.0×10⁷ cells per mouse. These animals were randomly divided into two groups of seven each for treatment with Compound A′ or vehicle. Compound A′ was formulated in the same manner, and mice were treated by oral application of 12.5 mg/kg Compound A′ or vehicle, once daily for 21 days (7-27 days after ATL cell inoculations). The therapeutic efficacy of Compound A′ was evaluated according to survival times.

4.3 Soluble IL-2 Receptor (sIL2R) Measurement

The concentration of human soluble IL-2 receptor (sIL2R) in mouse serum was measured by enzyme-linked immunosorbent assay (ELISA) using a human sIL2R immunoassay kit (R&D Systems, Minneapolis, Minn.) according to the manufacturer's instructions. All expressed values were averages of triplicate experiments.

### 4.4 Statistical Analysis

Differences between groups regarding the percentage of ATL cells in mouse liver and bone marrow cell suspensions, and human sIL2R concentrations in mouse sera, were examined using a Mann-Whitney U test. Mouse survival analyses were done by Kaplan-Meier method, and survival curves were compared using the log-rank test. All analyses were performed using SPSS Statistics 17.0 software (SPSS Inc., Chicago, Ill.). In this study, P<0.05 was considered significant.

### 4.5. In-Vivo Results

### 4.5.1 Compound A′ Treatment Reduced ATL Cells in Mouse Liver

Twenty-five days after ATL cell inoculation, the percentage of ATL cells (human CD45-positive, CD4-positive, and CD8-negative) in a liver cell suspension of control NOG mouse 1 was 10.6% (i.e., 19.1% [human CD45 positive cells]×55.3% [CD4 positive, but CD8 negative cells]=10.6%). In control NOG mice numbers 2, 3, 4, 5, 6, 7, and 8; and in Compound A′-treated NOG mice numbers 1, 2, 3, 4, 5, 6, 7, and 8, the percentages of ATL cells in liver cell suspensions, calculated in the same manner, were 8.1, 24.0, 20.5, 25.1, 15.7, 33.6, and 24.7%; and 0.9, 1.6, 1.0, 0.4, 2.2, 1.1, 0.6, and 0.5%, respectively. Thus, Compound A′ treatment significantly decreased the percentage of ATL cells infiltrating the liver of mice inoculated with ATL cells (P=0.001).

### 4.5.2 Compound A′ Treatment Reduced ATL Cells in Mouse Bone Marrow

The percentage of ATL cells (human CD45-positive, CD4-positive, and CD8-negative) in bone marrow cells of control NOG mouse 1 was 2.26% (i.e., 3.3% [human CD45 positive cells]×68.6% [CD4 positive, but CD8 negative cells]=2.26%). In control NOG mice 2, 3, 4, 5, 6, 7, and 8, and in Compound A′-treated NOG mice 1, 2, 3, 4, 5, 6, 7, and 8, the percentage of ATL cells in bone marrow cell suspensions, calculated in the same manner, were 1.18, 0.18, 1.31, 0.81, 1.12, 0.35, and 1.12%; and 0.01, 0.02, 0.01, 0.02, 0.01, 0.01, 0.72, and <0.01%, respectively. Thus, Compound A′ treatment significantly decreased the percentage of ATL cells infiltrating the bone marrow of these mice inoculated with ATL cells (P=0.002).

4.5.3 Soluble IL2R Concentrations in Mice with or without Compound A′ Therapy

The concentrations of human sIL2R in the serum of ATL cell-bearing control NOG mice 1-8 were 322.0, 323.6, 293.0-361.3×10³ pg/mL (mean, median, range), and those of Compound A′-treated NOG mice 1-8 were 84.5, 84.9, 69.0-97.4×10³ pg/mL. This difference was statistically significant (P=0.001). Thus, Compound A′ significantly reduced serum levels of human sIL2R in mice.

### 4.5.4 Compound A′ Induced a Prolongation of Survival Time in ATL Cell-Bearing Mice

Thirty-eight days after ATL cell inoculation, Compound A′-treated mice were all alive (n=7), but vehicle treated control mice were all dead (n=7) (P<0.001). Toxicity attributable to Compound A′ was not observed in any of the mice during the study period. Thus, a Compound A′-treated mouse group inoculated with ATL cells displayed a significant prolongation of survival time compared with untreated controls.

### 4.6 Summary and Conclusion

Compound A′ possesses significant therapeutic efficacies in a ATL mouse model, in which S-YU tumor cells survived and proliferated in a murine microenvironment.

Compound A′ shows strong potential as a novel treatment for patients with ATL.

The data demonstrate a significant decrease in ATL cell infiltration of the liver, bone marrow, and serum human soluble interleukin-2 levels (reflecting the ATL tumor burden), compared to untreated mice. In a separate experiment, Compound A′-treated, ATL-bearing mice demonstrated a significantly prolonged survival compared to control ATL-bearing mice. Of note, S-YU cells which were used for the in vivo studies showed the lowest sensitivity compared to 4 additionally tested ATL cell lines indicating that corresponding in vivo testing of these 4 additional cell lines may even result in superior efficacy data.

1-20. (canceled)

21: A method for treatment and/or prophylaxis of lymphoma comprising administering to a subject in need thereof an effective amount of a pharmaceutical combination comprising 4-(4-Fluoro-2-methoxyphenyl)-N-{3-[(S-methylsulfonimidoyl)methyl]phenyl}-1,3,5-triazin-2-amine of formula I or a physiologically acceptable salt or an enantiomer thereof, 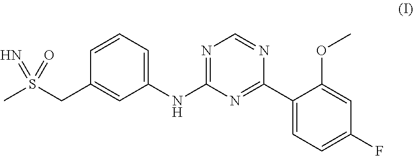 [SMILES: COc1cc(F)ccc1-c1ncnc(Nc2cccc(CS(C)(=N)=O)c2)n1]
and at least one or more further active ingredients.

22: A method for treatment and/or prophylaxis of lymphoma comprising administering to a subject in need thereof an effective amount of a pharmaceutical composition comprising 4-(4-Fluoro-2-methoxyphenyl)-N-{3-[(S-methylsulfonimidoyl)methyl]phenyl}-1,3,5-triazin-2-amine of formula I or a physiologically acceptable salt or an enantiomer thereof, 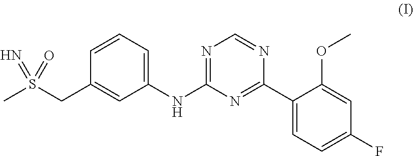 [SMILES: COc1cc(F)ccc1-c1ncnc(Nc2cccc(CS(C)(=N)=O)c2)n1]
and at least one inert, nontoxic, pharmaceutically suitable adjuvant.

23-26. (canceled)

27: A method for treatment and/or prophylaxis of lymphoma using an effective amount of 4-(4-Fluoro-2-methoxyphenyl)-N-{3-[(S-methylsulfonimidoyl)methyl]phenyl}-1,3,5-triazin-2-amine of formula I or a physiologically acceptable salt or an enantiomer thereof,

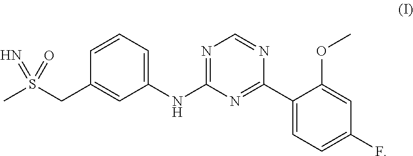 [SMILES: COc1cc(F)ccc1-c1ncnc(Nc2cccc(CS(C)(=N)=O)c2)n1]

28: The method according to claim 27, wherein
diffuse large B-cell lymphoma, mantle cell lymphoma, follicular lymphoma, Burkitt's lymphoma, adult T-cell lymphoma or Hodgkin's lymphoma is treated.

29: The method according to claim 28, wherein
diffuse large B-cell lymphoma is treated.

30: The method according to claim 28, wherein
adult T-cell lymphoma is treated.

31: The method according to claim 27, wherein the enantiomer
(+)-4-(4-Fluoro-2-methoxyphenyl)-N-{3-[(S-methylsulfonimidoyl)methyl]phenyl}-1,3,5-triazin-2-amine or a physiologically acceptable salt thereof is used.

32: The method according to claim 28, wherein the enantiomer
(+)-4-(4-Fluoro-2-methoxyphenyl)-N-{3-[(S-methylsulfonimidoyl)methyl]phenyl}-1,3,5-triazin-2-amine or a physiologically acceptable salt thereof is used.

33: The method according to claim 29, wherein the enantiomer
(+)-4-(4-Fluoro-2-methoxyphenyl)-N-{3-[(S-methylsulfonimidoyl)methyl]phenyl}-1,3,5-triazin-2-amine or a physiologically acceptable salt thereof is used.

34: The method according to claim 30, wherein the enantiomer
(+)-4-(4-Fluoro-2-methoxyphenyl)-N-{3-[(S-methylsulfonimidoyl)methyl]phenyl}-1,3,5-triazin-2-amine or a physiologically acceptable salt thereof is used.

35: The method according to claim 21, wherein
diffuse large B-cell lymphoma, mantle cell lymphoma, follicular lymphoma, Burkitt's lymphoma, adult T-cell lymphoma or Hodgkin's lymphoma is treated.

36: The method according to claim 35, wherein
diffuse large B-cell lymphoma is treated.

37: The method according to claim 35, wherein
adult T-cell lymphoma is treated.

38: The method according to claim 21, wherein the enantiomer
(+)-4-(4-Fluoro-2-methoxyphenyl)-N-{3-[(S-methylsulfonimidoyl)methyl]phenyl}-1,3,5-triazin-2-amine or a physiologically acceptable salt thereof is used.

39: The method according to claim 35, wherein the enantiomer
(+)-4-(4-Fluoro-2-methoxyphenyl)-N-{3-[(S-methylsulfonimidoyl)methyl]phenyl}-1,3,5-triazin-2-amine or a physiologically acceptable salt thereof is used.

40: The method according to claim 36, wherein the enantiomer
(+)-4-(4-Fluoro-2-methoxyphenyl)-N-{3-[(S-methylsulfonimidoyl)methyl]phenyl}-1,3,5-triazin-2-amine or a physiologically acceptable salt thereof is used.

41: The method according to claim 37, wherein the enantiomer
(+)-4-(4-Fluoro-2-methoxyphenyl)-N-{3-[(S-methylsulfonimidoyl)methyl]phenyl}-1,3,5-triazin-2-amine or a physiologically acceptable salt thereof is used.

42: The method according to claim 22, wherein
diffuse large B-cell lymphoma, mantle cell lymphoma, follicular lymphoma, Burkitt's lymphoma, adult T-cell lymphoma or Hodgkin's lymphoma is treated.

43: The method according to claim 42, wherein
diffuse large B-cell lymphoma is treated.

44: The method according to claim 42, wherein
adult T-cell lymphoma is treated.

45: The method according to claim 22, wherein the enantiomer
(+)-4-(4-Fluoro-2-methoxyphenyl)-N-{3-[(S-methylsulfonimidoyl)methyl]phenyl}-1,3,5-triazin-2-amine or a physiologically acceptable salt thereof is used.

46: The method according to claim 42, wherein the enantiomer
(+)-4-(4-Fluoro-2-methoxyphenyl)-N-{3-[(S-methylsulfonimidoyl)methyl]phenyl}-1,3,5-triazin-2-amine or a physiologically acceptable salt thereof is used.

47: The method according to claim 43, wherein the enantiomer
(+)-4-(4-Fluoro-2-methoxyphenyl)-N-{3-[(S-methylsulfonimidoyl)methyl]phenyl}-1,3,5-triazin-2-amine or a physiologically acceptable salt thereof is used.

48: The method according to claim 44, wherein the enantiomer
(+)-4-(4-Fluoro-2-methoxyphenyl)-N-{3-[(S-methylsulfonimidoyl)methyl]phenyl}-1,3,5-triazin-2-amine or a physiologically acceptable salt thereof is used.
